(19) United States
(12) Patent Application Publication
(10) Pub. No.: US 2005/0153360 A1
(43) Pub. Date: Jul. 14, 2005

(54) Diagnostic method for stroke

(76) Inventors: Denis Francois Hochstrasser, Geneva (CH); Jean-Charles Sanchez, Geneva (CH); Elisabeth Guillaume, Annemasse (FR); Laure Allard, Gaillard (FR)

Correspondence Address: ARENT FOX PLLC, 1675 BROADWAY, NEW YORK, NY, 10019, US

(21) Appl. No.: 10/909,761

(22) Filed: Aug. 2, 2004

Related U.S. Application Data
(63) Continuation of application No. PCT/EP03/01462, filed on Feb. 13, 2003.

(30) Foreign Application Priority Data
Feb. 18, 2002 (GB) 02 03768.7

(51) Int. Cl. G01N 33/53; G01N 33/00

(52) U.S. Cl. 435/7.1; 436/86

(57) ABSTRACT

The present invention provides a method of diagnosis of stroke or the possibility thereof in a subject suspected of suffering from stroke, comprising subjecting a sample of body fluid taken from the subject to mass spectrometry to determine a test amount of a polypeptide in the sample, wherein the polypeptide is differentially contained in the body fluid of stroke-affected subjects and non-stroke-affected subjects, and has a molecular weight in the range of from 3000 to 30000 Da; and determining whether the test amount is consistent with a diagnosis of stroke. The test amount can also be used to determine the type of stroke that is diagnosed, in particular whether it is of the ischemic or hemorrhagic type. The invention also provides use of the polypeptides for diagnostic, prognostic and therapeutic applications. The invention further provides a kit for use in diagnosis of stroke, comprising a probe for receiving a sample of body fluid, and for placement in a mass spectrometer, thereby to determine a test amount of a polypeptide in the sample, wherein the polypeptide is differentially contained in the body fluid of stroke-affected subjects and non-stroke-affected subjects, and has a molecular weight in the range of from 3000 to 30000 Da.

### CROSS REFERENCE TO RELATED APPLICATION(S)

The present application is a continuation of International Patent Application No. PCT/EP03/01462, filed Feb. 13, 2003, published in English on Aug. 21, 2003 as International Patent Publication No. WO03/069346, which claims priority to British Patent Application No. 02 03768.7, filed Feb. 18, 2002, all of which are incorporated by reference herein in their entireties.

### BACKGROUND OF THE INVENTION

This invention relates to a diagnostic method for stroke.

Stroke has the third highest death rate in industrial countries. It is caused either by bleeding in the brain from a ruptured blood vessel (hemorrhagic stroke) or by obstruction of a blood vessel in the brain (ischemic or thrombotic stroke). Stroke results from either a permanent or a transient reduction in cerebral blood flow. This reduction in flow is, in most cases, caused by the arterial occlusion due to either an embolus or a local thrombosis. Depending on the localization of brain injury and the intensity of necrosed neurons, stroke symptoms can become a life handicap for patients and the death rate from stroke events approaches 30%.

Recently, S100B was described as a potential biochemical marker for stroke diagnosis, see U. Missler et al., “S100 protein and neuron-specific enolase concentrations in blood as indicators of infarct volume and prognosis in acute ischemia stroke,” Stroke 1997; 28:1956-60. However, S100B has also been reported as a useful marker for early detection of metastases of melanoma and cerebral complications from head injury and cardiac surgery. Thus, the sensitivity and specificity of the S100B test were limited to 44% and 67%, respectively, see M. Takahashi et al., “Rapid and sensitive immunoassay for the measurement of serum S100B using isoform-specific monoclonal antibody,” Clin. Chem. 1999; 45: 1307-11. Development of new stroke markers would help clinicians to establish early diagnosis.

WO 01/42793, which corresponds to U.S. patent application Ser. No. 10/165,127, relates to a diagnostic assay for stroke in which the concentration of heart or brain fatty acid binding protein (H-FABP or B-FABP) is determined in a sample of body fluid.

U.S. Pat. No. 6,225,047 describes the use of retentate chromatography to generate difference maps, and in particular a method of identifying analyses that are differentially present between two samples. One specific method described therein is laser desorption mass spectrometry.

WO 01/25791 describes a method for aiding a prostate cancer diagnosis, which comprises determining a test amount of a polypeptide marker, which is differentially present in samples of a prostate cancer patient and a subject who does not have prostate cancer. The marker may be determined using mass spectrometry, and preferably laser desorption mass spectrometry.

Development of new non-invasive stroke markers for body fluids and new methods of determining the markers would help clinicians to establish early diagnosis. This problem has now been solved by the present invention.

### SUMMARY OF THE INVENTION

The present invention provides a method for diagnosis of stroke or the possibility thereof in a subject suspected of suffering from stroke, which comprises subjecting a sample of body fluid taken from the subject to mass spectrometry, thereby to determine a test amount of at least one (one or more) polypeptide in the sample, wherein the polypeptide is differentially contained in the body fluid of stroke-affected subjects and non-stroke-affected subjects, and has a molecular weight in the range of from 3000 to 30,000 Da; and determining whether the test amount is consistent with a diagnosis of stroke. The test amount can also be used to determine the type of stroke that is diagnosed, in particular whether it is of the ischemic or hemorrhagic type.

The invention also provides use of a polypeptide which is differentially contained in a body fluid of stroke-affected subjects and non-stroke-affected subjects, the polypeptide having a molecular weight in the range of from 3000 to 30,000 Da and being determinable by mass spectrometry, for diagnostic, prognostic and therapeutic applications.

The invention further provides a kit for use in diagnosis of stroke, comprising a probe for receiving a sample of body fluid, and for placement in a mass spectrometer, thereby to determine a test amount of a polypeptide in the sample, wherein the polypeptide is differentially contained in the body fluid of stroke-affected subjects and non-stroke-affected subjects, and has a molecular weight in the range of from 3000 to 30,000 Da.

### BRIEF DESCRIPTION OF THE DRAWINGS

FIG. 1 (A and B) is a spectral view of plasma from four hemorrhagic stroke patients (H 1-4) and four control samples (CTRL 1-4) using laser desorption/ionization mass spectrometry, in the molecular weight range of 3750 to 4750 Da;

FIG. 2 (A and B) is a view corresponding to FIG. 1, but in the molecular weight range of 5000 to 11,000 Da;

FIG. 3 (A and B) is a view corresponding to FIG. 1, but in the molecular weight range of 12000 to 30,000 Da;

FIG. 4 (A and B) is a spectral view of plasma from four ischemic stroke patients (I 1-4) and four control samples (CTRL 1-4) using laser desorption/ionization mass spectrometry, in the molecular weight range of 3750 to 4750 Da;

FIG. 5 (A and B) is a view corresponding to FIG. 4, but in the molecular weight range of 5000 to 11,000 Da;

FIG. 6 (A and B) is a view corresponding to FIG. 4, but in the molecular weight range of 12000 to 30,000 Da;

FIG. 7 (A, B and C) is a spectral view of plasma from four stroke patients (identified as 155 stroke, 184 stroke, 194 stroke and 195 stroke) and four control samples (identified as 380 neg, 386 neg, 387 neg and 390 neg) using laser desorption/ionization mass spectrometry, in the molecular weight range of about 4300 to 5000 Da;

FIG. 8 (A, B and C) is a view corresponding to FIG. 7, but in the molecular weight range of about 5000 to 8000 Da; and

FIG. 9 (A, B and C) is a view corresponding to FIG. 7, but in the molecular weight range of 10000 to 16,000 Da.

In the Figures, the horizontal axis represents molecular weight in Da (m/z ratio) and the vertical axis represents signal intensity, i.e. amount of material having the given molecular weight.

### DESCRIPTION OF PREFERRED EMBODIMENTS

The invention provides a method of diagnosis of stroke or the possibility thereof in a subject suspected of suffering from stroke. A sample of body fluid taken from the subject is subjected to mass spectrometry, to determine the presence or absence in the sample of a polypeptide marker which is differentially contained in the body fluid of stroke-affected subjects and non-affected subjects. The polypeptide marker has a molecular weight in the range of from 3000 to 30,000 Da, preferably from 3900 to 29,000 Da, and the presence or absence of the marker is indicative of stroke. A particular feature of the invention is that the presence or absence of certain markers can be used to determine whether a diagnosed stroke is of the ischemic or hemorrhagic type.

The term polypeptide includes proteins and protein fragments, as well as peptides modified by the addition of non-peptide residues, e.g. carbohydrates, phosphates, sulfates or any other post-translational modification.

The sample may be adsorbed on a probe under conditions which allow binding between the polypeptide and adsorbent material on the probe. The adsorbent material preferably comprises a metal chelating group complexed with a metal ion, and a preferred metal is copper. Prior to detecting the polypeptide, unbound or weakly bound materials on the probe may be removed with a washing solution, thereby enriching the polypeptide in the sample. The sample is preferably adsorbed on a probe having an immobilized metal affinity capture (IMAC) or a strong anion exchange (SAX) surface capable of binding the polypeptide. The sample may be also adsorbed on a probe having hydrophobic, strong anionic or weak cationic exchange surfaces under conditions which allow binding of the polypeptides. The probe may consist of a strip having several adsorbent wells, and be inserted into the spectrometer, then movable therein so that each well is in turn struck by the ionizing means (e.g. laser) to give a spectrometer reading. The polypeptide is preferably determined by surface-enhanced laser desorption/ionization (SELDI) and time of flight mass spectrometry (TOF-MS).

In principle, any body fluid can be used to provide a sample for diagnosis, but preferably the body fluid is cerebrospinal fluid (CSF), plasma, serum, blood, urine or tears.

In one embodiment of the invention, at least one (one or more) polypeptide having a respective molecular weight of about 3900, about 3970, about 3990, about 6945, about 10,070, about 14,040 and/or about 28,000 Da, respectively, is determined, and increase or reduction, relative to a control, of a peak corresponding to the polypeptide is indicative of stroke. The 3900 peak is mostly higher than the 3970 and 3990 peaks in stroke plasma samples.

In another embodiment of the invention, at least one (one or more) polypeptide having a respective molecular weight of about 5920, about 6660 and/or about 7770 Da is determined, and increase or reduction, relative to a control, of a peak corresponding to the polypeptide is indicative of stroke.

In a further embodiment of the invention, at least one (one or more) polypeptide having a respective molecular weight of about 3900, about 3970, about 3990, about 14,040 and/or about 28,000 Da is determined, and increase or reduction, relative to a control, of a peak corresponding to the polypeptide is used to indicate whether a diagnosed stroke is of the ischemic or hemorrhagic type.

Generally, the following observations, separately or in any combination, are characteristic of hemorrhagic stroke (when compared to a control): decrease of a peak at about 3970 Da; decrease of a peak at about 5920 and/or about 10,070 Da; increase of a peak at about 6660 and/or about 6945 and/or about 7770 Da; and decrease of a peak at about 14,040 and/or about 28,000 Da.

Generally, the following observations, separately or in any combination, are characteristic of ischemic stroke (when compared to a control): a peak at about 3970 Da greater than a peak at about 3990 Da, but both lower than a peak at about 3900 Da; decrease of a peak at about 5920 and/or about 10,070 Da; increase of a peak at about 7770 Da; and no decrease of peaks at about 14,040 and/or about 28,000 Da.

In a further embodiment of the invention, at least one (one or more) polypeptide having a respective molecular weight of about 4475, about 4634 and/or about 4797 Da is determined, and reduction, relative to a control, of a peak corresponding to the polypeptide is indicative of stroke.

In a still further embodiment, at least one (one or more) polypeptide having a respective molecular weight of about 6441 and/or about 6643 Da is determined, and increase, relative to a control, of a peak corresponding to the polypeptide is indicative of stroke.

In a yet further embodiment, at least one (one or more) polypeptide having a respective molecular weight of about 11,530 and/or about 11,712 Da is determined, and reduction, relative to a control, of a peak corresponding to the polypeptide is indicative of stroke.

According to the invention, a diagnosis of stroke may be made from measurement at a single molecular weight or at any combination of two or more molecular weights, which may, for example, be selected from those molecular weights mentioned above.

Measurement of the molecular weight of the polypeptide or polypeptides is effected in the mass spectrometer. The molecular weights quoted above can be measured with an accuracy of better than 1%, and preferably to within about 0.1%. The term “about” in connection with molecular weights therefore means within a variation of about 1%, preferably within about 0.1%, above or below the quoted value.

The invention also relates to the use of a polypeptide which is differentially contained in a body fluid of stroke-affected subjects and non-stroke-affected subjects, the polypeptide having a molecular weight in the range of from 3000 to 30,000 Da and being determinable by mass spectrometry, for diagnostic, prognostic and therapeutic applications. This may involve the preparation and/or use of a material which recognizes, binds to or has some affinity to the above-mentioned polypeptide. Examples of such materials are antibodies and antibody chips. The term “antibody” as used herein includes polyclonal antiserum, monoclonal antibodies, fragments of antibodies such as Fab, and genetically engineered antibodies. The antibodies may be chimeric or of a single species. The above reference to “prognostic” applications includes making a determination of the likely course of a stroke by, for example, measuring the amount of the above-mentioned polypeptide in a sample of body fluid. The above reference to “therapeutic” applications includes, for example, preparing materials which recognize, bind to or have affinity to the above-mentioned polypeptides, and using such materials in therapy. The materials may in this case be modified, for example by combining an antibody with a drug, thereby to target the drug to a specific region of the animal to be treated.

The methodology of this invention can be applied to the diagnosis of any kind of stroke. Body fluid samples are prepared from stroke-affected and non-stroke-affected subjects. The samples are applied to a probe having a surface treated with a variety of adsorbent media, for differential retention of peptides in the sample, optionally using washing liquids to remove unbound or weakly bound materials. If appropriate, energy-absorbing material can also be applied. The probe is then inserted into a mass spectrometer, and readings are taken for the various sample/adsorbent combinations using a variety of spectrometer settings. Comparison of the affected and non-affected samples under a given set of conditions reveals one or more polypeptides which are differentially expressed in the affected and non-affected samples. The presence or absence of these polypeptides can then be used in the testing of a fluid sample from a subject under the same conditions (adsorbent, spectrometer settings etc.) to determine whether or not the subject is affected. Furthermore, by comparing, on the one hand, hemorrhagic stroke samples with a control, and, on the other hand, ischemic stroke samples with a control, it is possible to discriminate between the possibility of hemorrhagic stroke or ischemic stroke by testing a body fluid sample from a patient under the same conditions.

The reference to “presence” or “absence” of a polypeptide found throughout the specification should be understood to mean simply that there is a significant difference in the amount of a polypeptide which is detected in the affected and non-affected sample. Thus, the “absence” of a polypeptide in a test sample may include the possibility that the polypeptide is actually present, but in a significantly lower amount than in a comparative test sample. According to the invention, a diagnosis can be made on the basis of the presence or absence of a polypeptide, and this includes the presence of a polypeptide in a significantly lower or significantly higher amount with reference to a comparative test sample.

The use of the phrases “differentially expressed,” “differentially contained,” and “differentially present” should be understood to mean that the amount of the polypeptide detected in the stroke-affected sample is different in comparison to the amount of the polypeptide detected in the non-affected or control sample. The difference in the amount of the polypeptide may be an increase or decrease relative to the amount identified in the non-affected or control sample.

The use of the term “a” with polypeptide should be understood to include “at least one” polypeptide or “one or more” polypeptides.

The following Examples illustrate the invention.

### EXAMPLE 1

The objective of the present study was to detect specific polypeptides in body fluids (cerebrospinal fluid, plasma and others) of stroke-affected patients. Samples were analyzed by the Surface Enhanced Laser Desorption Ionization (SELDI) Mass Spectroscopy (MS) technology. This technology encompasses micro-scale affinity capture of proteins by using different types of retentate chromatography and then analysis by time of flight mass spectrometry. Difference maps are thus generated each corresponding to a typical protein profiling of given samples that were analyzed with a Ciphergen Biosystem PBS II mass spectrometer (Freemont, Calif., USA). Differential expressed peaks were identified when comparing spectra generated in a group of plasma samples from stroke-affected patients with a control group of non-affected patients.

The SELDI analysis was performed using 2 μl of crude human plasma samples in order to detect specific polypeptides with metal affinity. An immobilized copper affinity array (IMAC-Cu⁺⁺) was employed in this approach to capture proteins with affinity for copper to select for a specific subset of proteins from the samples. Captured proteins were directly detected using the PBSII Protein Chip Array reader (Ciphergen Biosystems, Freemont, Calif., USA).

The following protocol was used for the processing and analysis of ProteinChip arrays using Chromatographic TED-Cu(II) adsorbent array. TED is a (tris(carboxymethyl) ethylenediamine-Cu) adsorbent coated on a silicon oxide-coated stainless steel substrate.
The surface was first loaded with 10 μl of 100 mM copper sulfate to each spot and incubated for 15 minutes in a wet chamber.
The chip was thereafter washed by two quick rinses with deionized water for about 10 seconds to remove the excess unbound copper.
Before loading the samples, the I-MAC 3 array was equilibrated once with 5 μl of PBS NaCl 0.5 M for 5 minutes.
After removing the equilibration buffer, 3 μl of the same buffer were added before applying 2 μl of plasma. The chip was incubated for 20 minutes in a wet chamber.
The samples were thereafter removed and the surface was washed three times with the equilibration buffer (5 minutes each).
Two quick final rinses with water were performed.
The surface was allowed to air dry, followed by the addition of 0.5 μl of saturated sinapinic acid (SPA, Ciphergen Biosystem) prepared in 50% acetonitrile, 0.5% trifluoroacetic acid.
The chip was air dried again before analysis of the retained protein on each spot with laser desorption/ionization time-of-flight mass spectrometry.
The protein chip array was inserted into the instrument and analyzed once the appropriate detector sensitivity and laser energy have been established to automate the data collection.
The obtained spectra were analyzed with the Biomark Wizard software (Ciphergen Biosystems, Freemont, Calif., USA) running on a Dell Dimension 4100 PC. It generates consistent peak sets across multiple spectra.

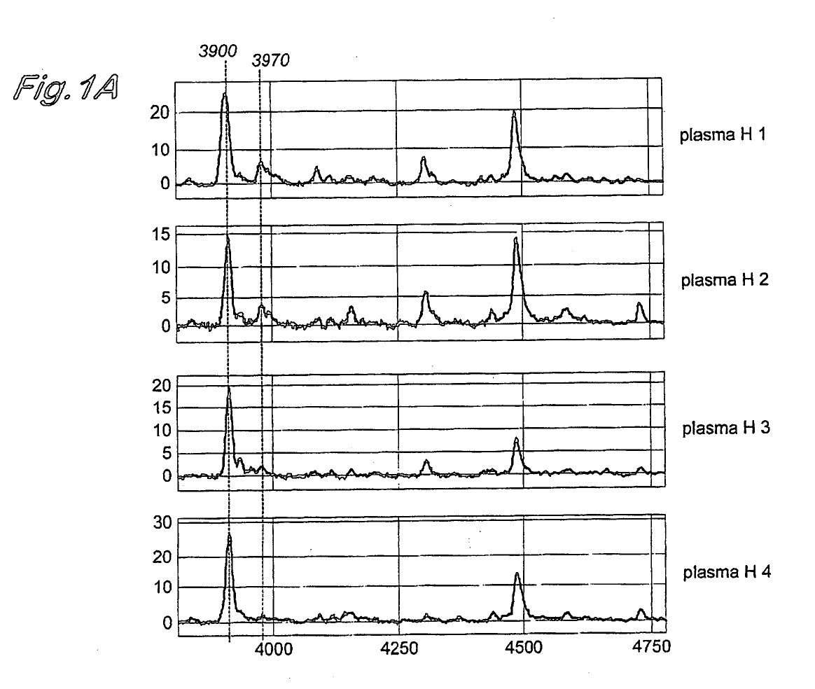

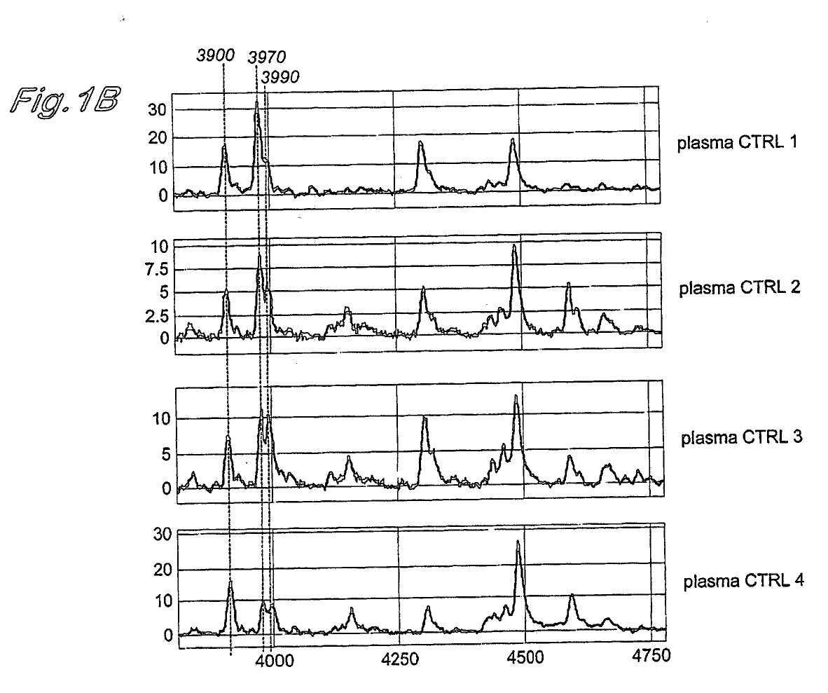

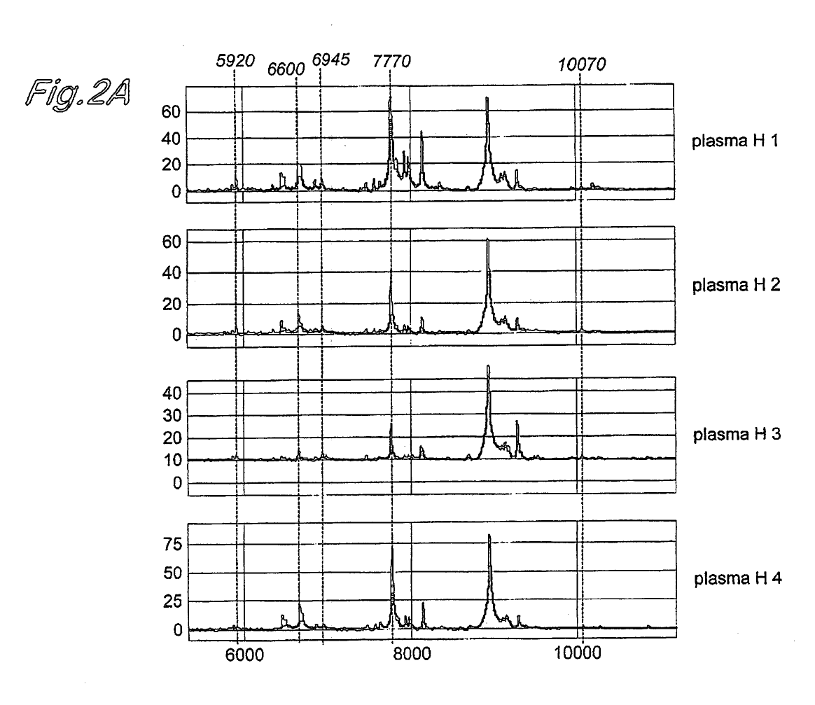

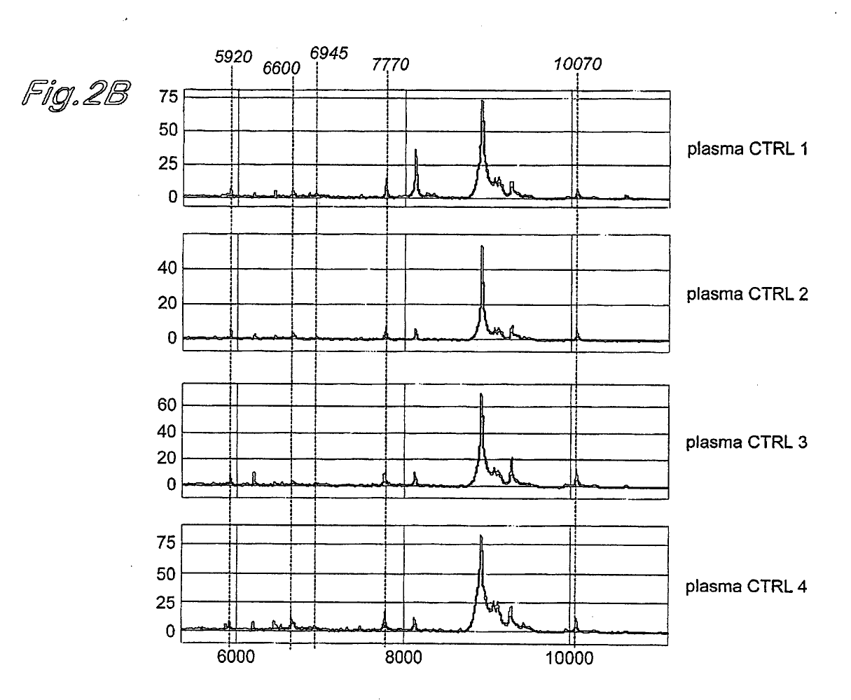

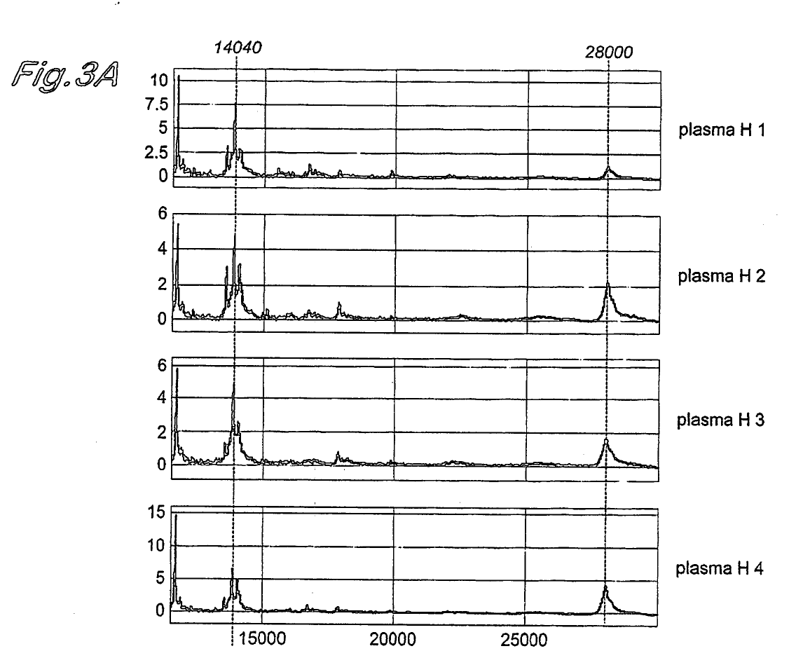

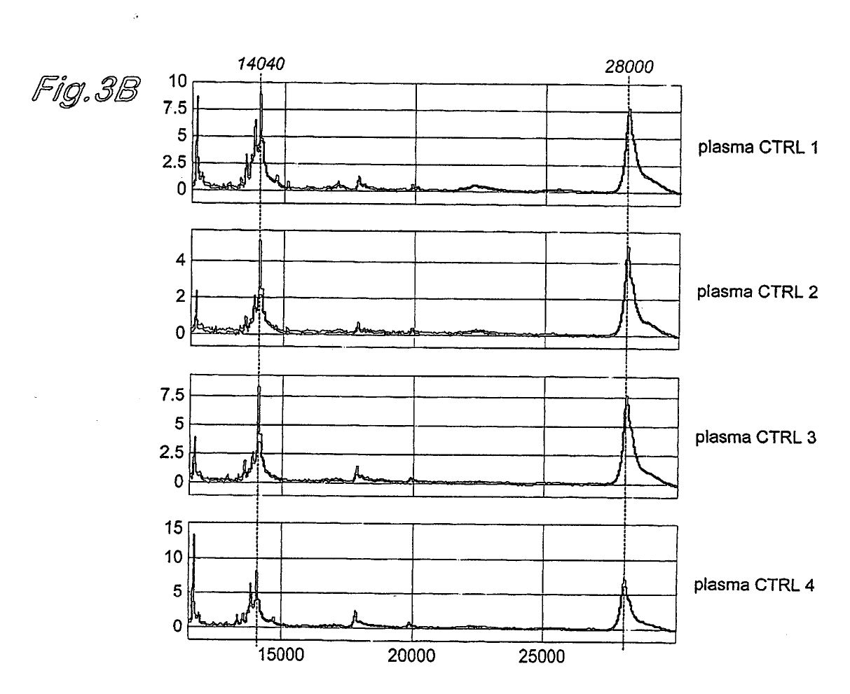

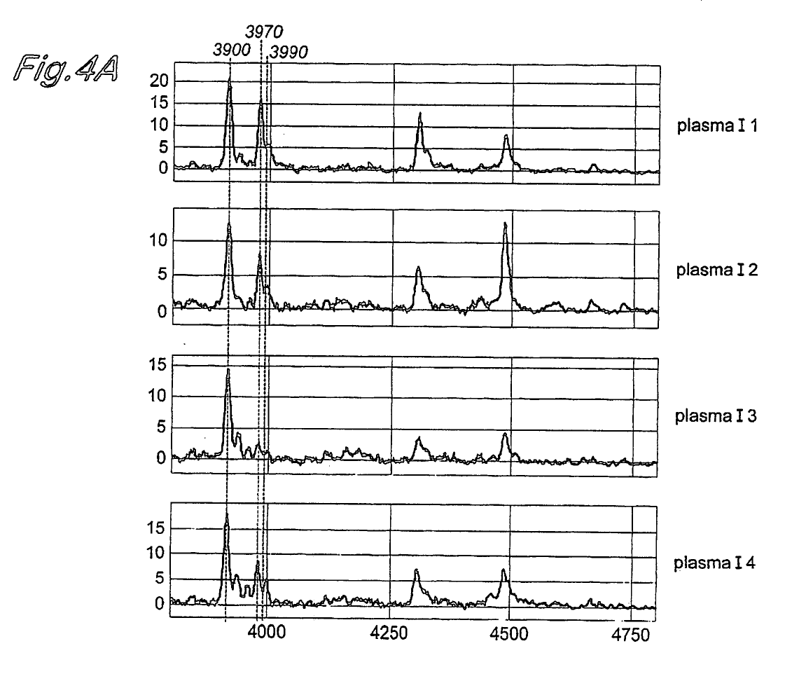

The results of the above tests on four plasma samples from hemorrhagic stroke patients (plasma H 1-4) and four plasma samples from non-affected subjects (plasma CTRL 1-4) are shown in FIGS. 1 to 3. FIG. 1 shows the strong decrease of a peak around 3970 Da in hemorrhagic samples as compared to healthy ones. In the control samples it forms a pair with a peak at about 3990 Da, but in the hemorrhagic stroke samples the pair have nearly disappeared behind the peak at about 3900 Da, which has been strongly increased. FIG. 2 highlights the decrease of two peaks around 5920 and 10,070 Da in hemorrhagic stroke samples as compared to healthy ones. FIG. 2 also shows the increase of peaks at about 6660, 6945 and 7770 Da in hemorrhagic stroke samples as compared to healthy ones. FIG. 3 shows a decreased intensity of peaks at about 14,040 and 28,000 Da in hemorrhagic stroke samples as compared to healthy ones.

### EXAMPLE 2

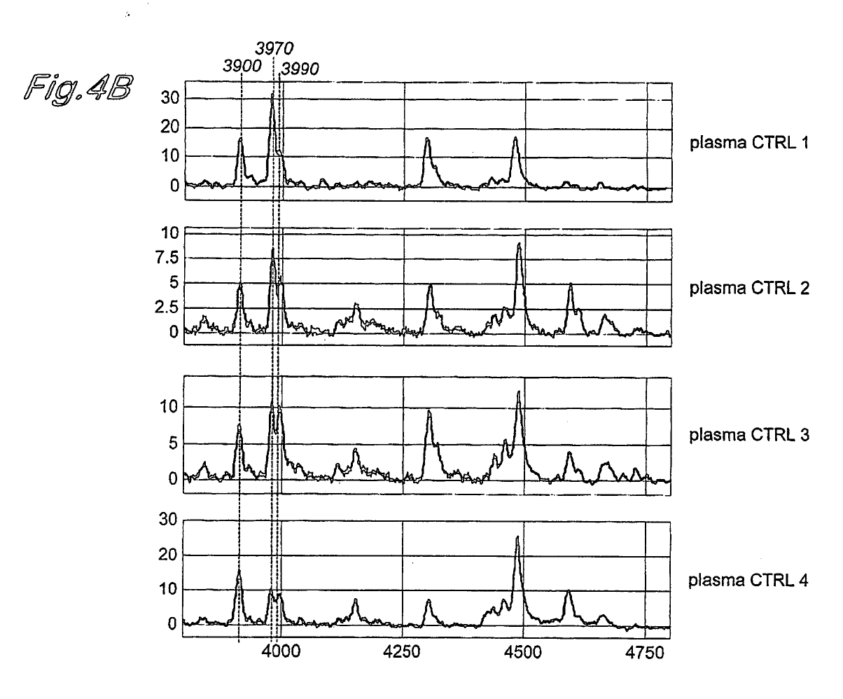

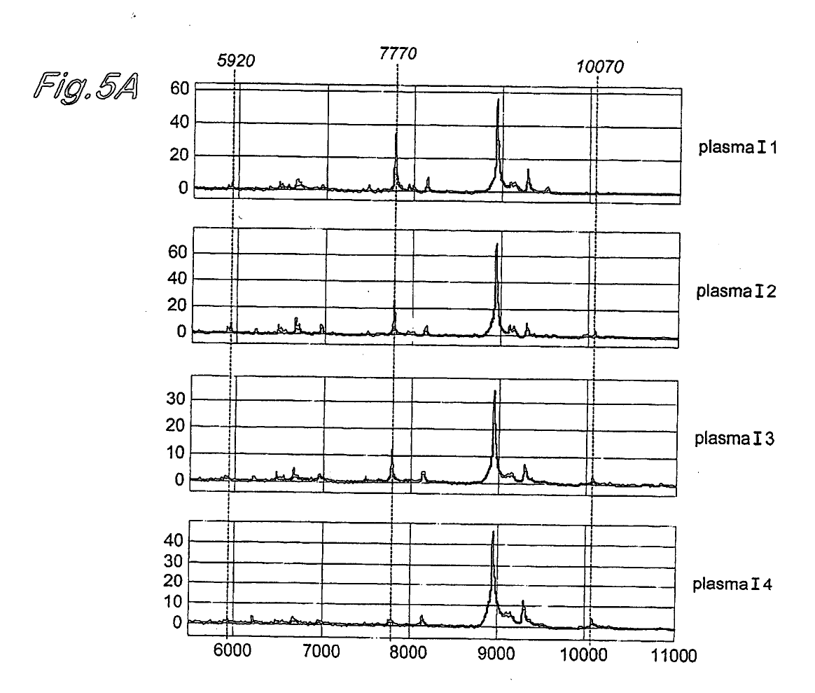

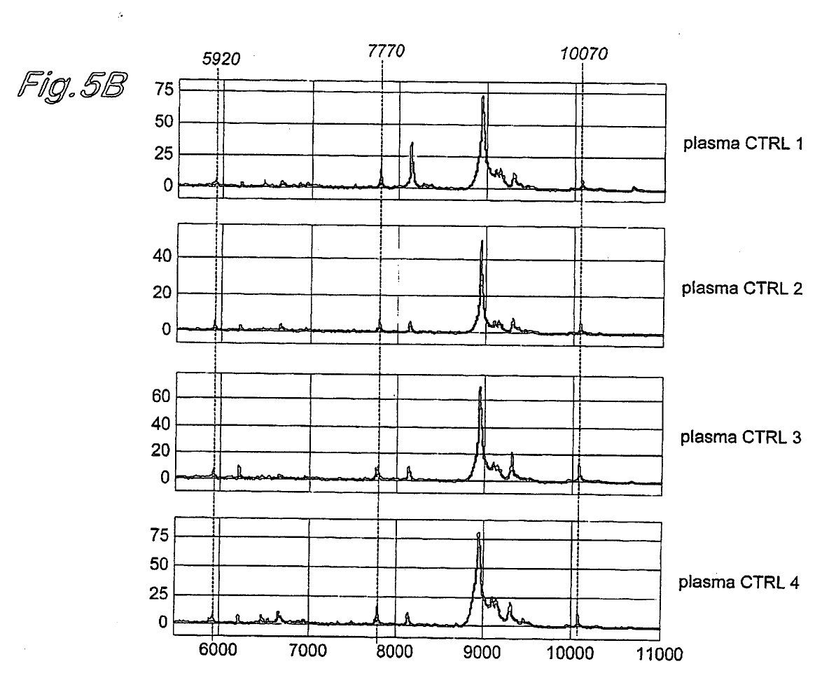

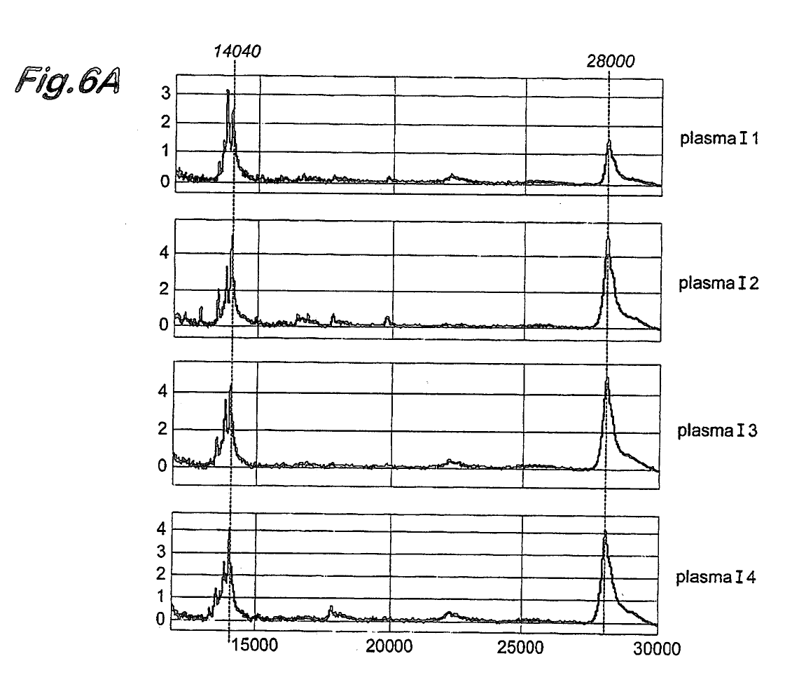

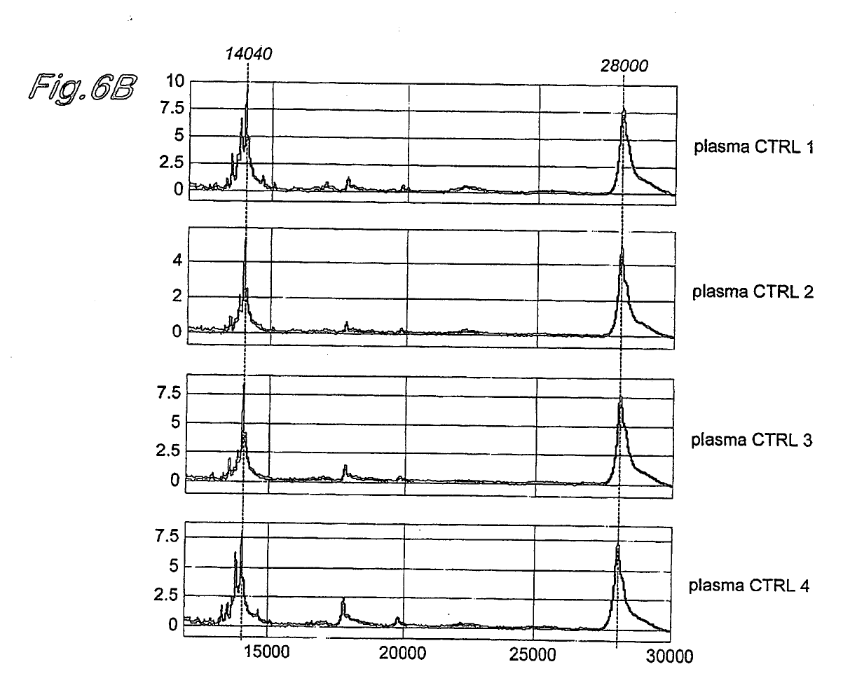

The procedure of Example 1 is repeated on four plasma samples from ischemic stroke patients (plasma I 1-4) and four plasma samples from non-affected subjects (plasma CTRL 1-4). The results are shown in FIGS. 4 to 6. FIG. 4 shows for the ischemic stroke samples a pair of peaks at 3970 and 3990 Da, where the 3970 peak is higher than the 3990 peak, but of a lower intensity than the 3900 peak, in contrast to the control samples. FIG. 5 highlights the decrease of two peaks around 5920 and 10,070 Da in ischemic stroke samples as compared to healthy ones. FIG. 5 also shows the 7770 peak increased in ischemic stroke samples, but to a lesser extent than in hemorrhagic stroke samples. FIG. 6 does not show any decrease of peaks around 14,040 and 28,000 Da between ischemic stroke samples and healthy samples, in contrast to the differences shown for hemorrhagic stroke samples in FIG. 3.

### EXAMPLE 3

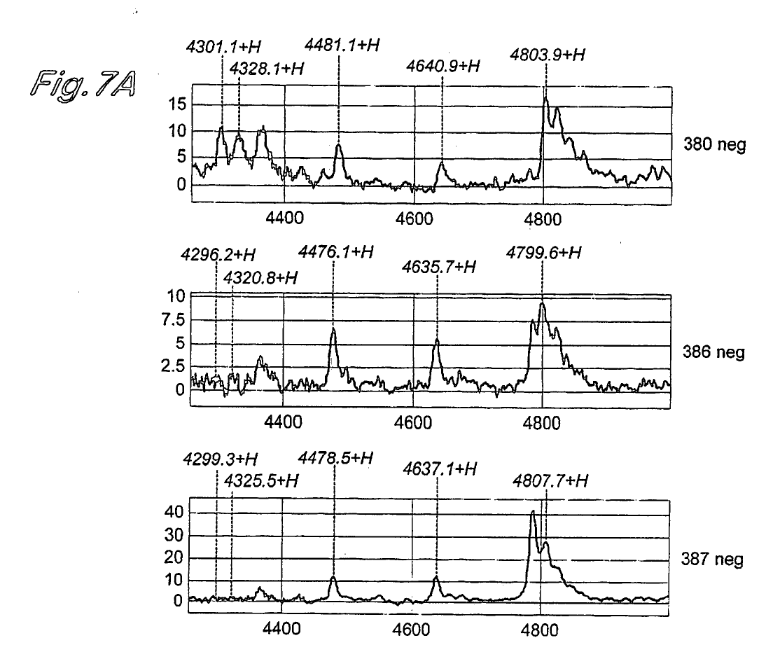

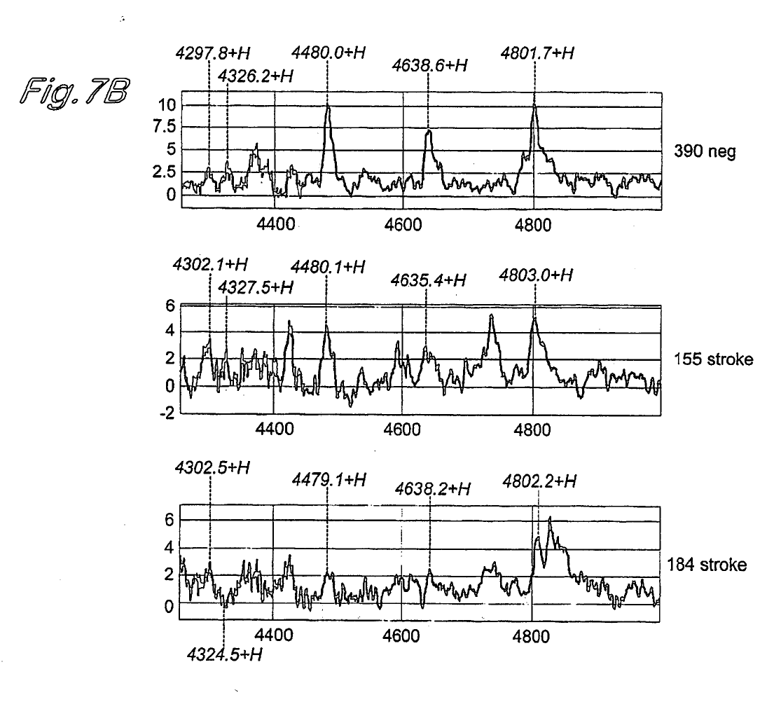

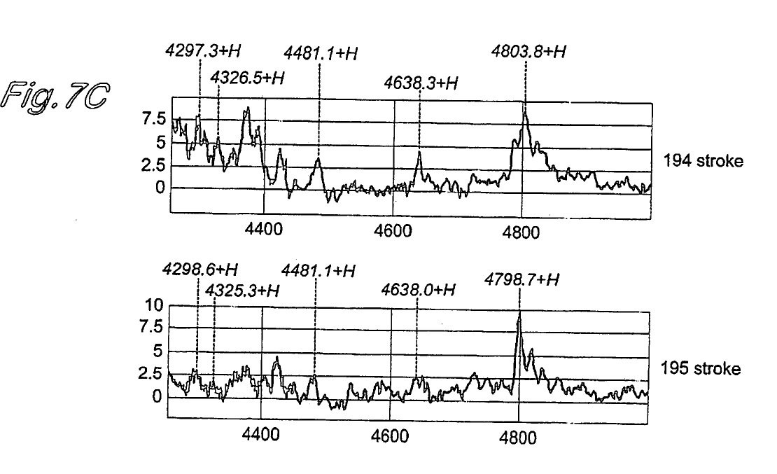

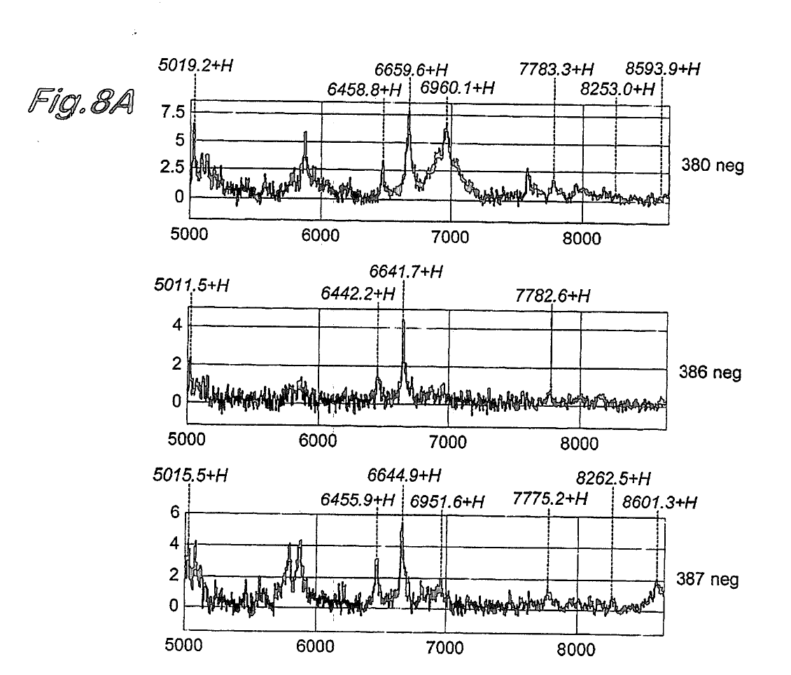

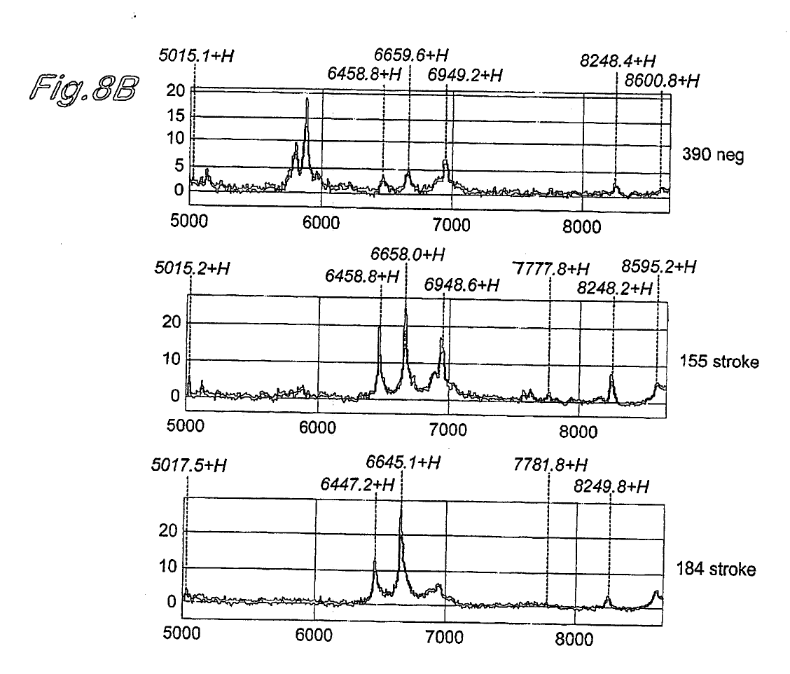

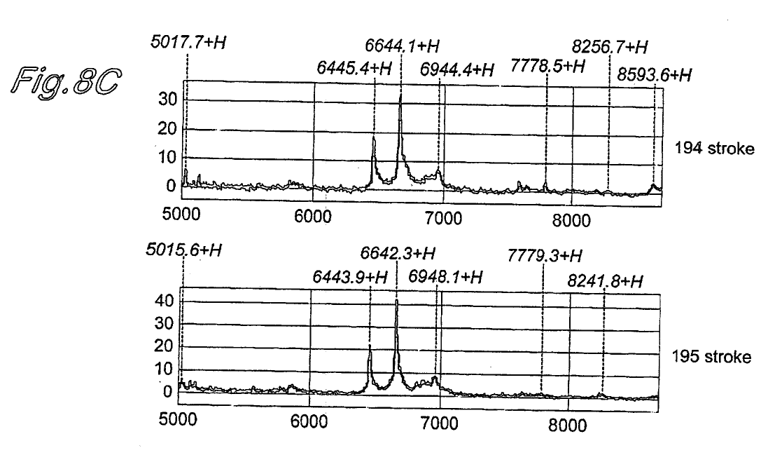

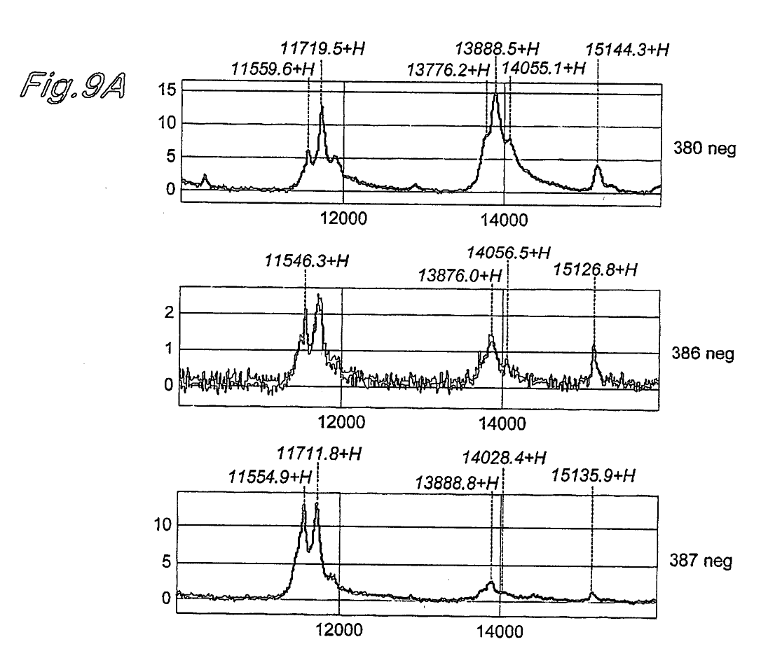

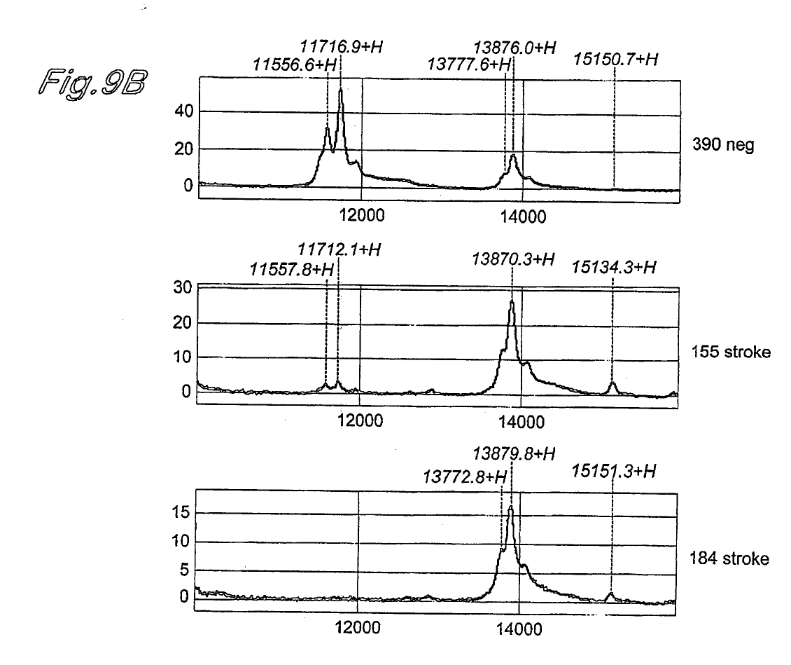

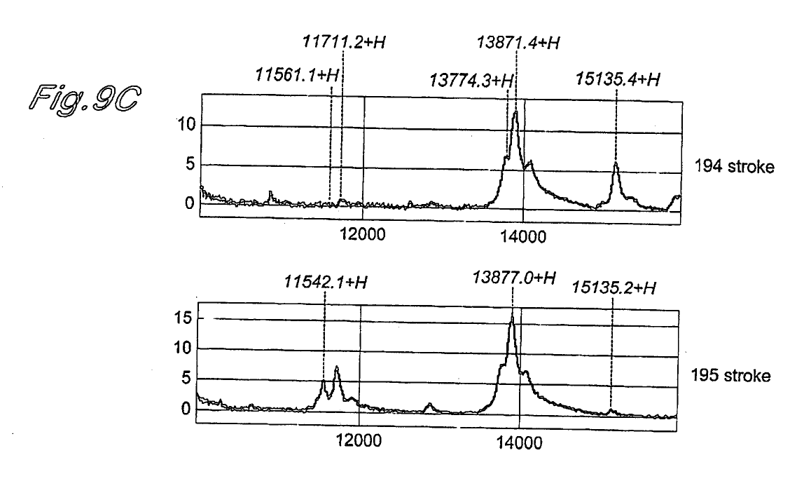

A comparative investigation between plasma samples coming from 21 stroke patients (including 10 hemorrhagic, 10 ischemic and 1 unknown type) and 21 healthy patients was carried out using the SELDI technology, in a similar way to the procedure of Example except for the variations mentioned hereafter. SAX ProteinChips (Ciphergen) and a SPA (Ciphergen) matrix were retained for the study. An example of 4 stroke spectra and 4 healthy patient spectra among the 42 tested is given in FIGS. 7 to 9. Using the Biomarker Wizard (Mann and Whitney statistical analysis), seven peaks appeared differentially expressed between stroke and healthy controls: a decrease of the signal of the peaks at 4475 Da, 4634 Da and 4797 Da is indicative of stroke with p values of 0.000138, 0.00224 and 0.0132 respectively. An increase of the peaks at 6443 Da and 6641 Da is indicative of stroke with p values of 0.08950 and 0.02134. And a decrease of the peaks at 11,530 Da and 11,712 Da, relative to a control, is indicative of stroke with p values of 0.00634 and 0.04034 respectively.

The following protocol was used for the processing and analysis of the SAX ProteinChips:
1. Outline each spot using a hydrophobic pen. Allow to air dry.
2. Apply 10 μl binding buffer (20 mM Tris−5 mM NaCl pH9.0) to each spot and incubate in a humidity chamber at room temperature for 5 minutes. Do not allow the spots to become dry.
3. Remove excess buffer from the spots without touching the active surface. Repeat steps 2 and 3 two more times.
4. Load 1 μl crude plasma sample+2 μl binding buffer (20 mM Tris−5 mM NaCl pH9.0).
5. Incubate in a humidity chamber for 30 minutes.
6. Wash each spot with 5 μl binding buffer (20 mM Tris−5 mM NaCl pH9.0) 5 times, followed by two quick washes with water (5 μl per wash).
7. Wipe dry around the spots. Apply 0.5 μl SPA saturated matrix (Ciphergen) to each spot while it is still moist, but not wet. Air dry. Apply a second 0.5 μl of SPA saturated matrix (Ciphergen) and air dry again before analysis of the retained protein on each spot with laser desorption/ionization time-of-flight mass spectrometry
8. The protein chip array was inserted into the instrument and analyzed once the appropriate detector sensitivity and laser energy have been established to automate the data collection.

Each of the above cited publications is herein incorporated by reference to the extent to which it is relied on herein.

1. A method for diagnosis of stroke or the possibility thereof in a subject suspected of suffering from stroke, comprising
subjecting a sample of body fluid taken from the subject to mass spectrometry to determine a test amount of a polypeptide in the sample, wherein the polypeptide is differentially contained in the body fluid of a stroke-affected subject and a non-stroke-affected subject, said polypeptide having a molecular weight selected from the group consisting of about 3900, about 3970, about 3990, about 6945, about 10,070, about 14,040, about 28,000, about 5920, about 6660, about 7770, about 4475, about 4634, about 4797, about 6441, about 6643, about 11,530 and about 11,712 Da; and
determining whether the test amount of the polypeptide is consistent with a diagnosis of stroke in the affected subject.

2. The method according to claim 1, wherein the presence of the polypeptide in the body fluid of a stroke-affected subject and the absence of the polypeptide in the body fluid of a non-stroke-affected subject is indicative of stroke.

3. The method according to claim 1, wherein the absence of the polypeptide in the body fluid of a stroke-affected subject and the presence of the polypeptide in the body fluid of a non-stroke-affected subject is indicative of stroke.

4. The method according to claim 1, wherein the mass spectrometry is laser desorption/ionization mass spectrometry.

5. The method according to claim 1, wherein the sample is adsorbed on a probe having an immobilized metal affinity capture (IMAC), hydrophobic, strong anionic or weak cationic exchange surface capable of binding the polypeptide.

6. The method according to claim 1, wherein the presence or absence of the polypeptide is determined by surface-enhanced laser desorption/ionization (SELDI) and time of flight mass spectrometry (TOF-MS).

7. The method according to claim 1, wherein the body fluid is cerebrospinal fluid, plasma, serum, blood or tears.

8. The method according to to claim 1, wherein a plurality of peptides is determined in the sample.

9. The method according to claim 1, wherein the test amount of polypeptide is used to determine whether a diagnosed stroke is of the ischemic or hemorrhagic type.

10. The method according to claim 1, wherein the polypeptide has a molecular weight selected from the group consisting of about 3900, about 3970, about 3990, about 6945, about 10,070, about 14,040, and about 28,000 Da, and wherein an increase or reduction, relative to a control, of a peak corresponding to the polypeptide is indicative of stroke.

11. The method according to claim 1, wherein the polypeptide has a molecular weight selected from the group consisting of about 5920, about 6660 and about 7770 Da, and an increase or reduction, relative to a control, of a peak corresponding to the polypeptide is indicative of stroke.

12. The method according to claim 1, wherein the polypeptide has a molecular weight selected from the group consisting of about 3900, about 3970, about 3990, about 14,040 and about 28,000 Da, and an increase or reduction, relative to a control, of a peak corresponding to the polypeptide is used to indicate whether a diagnosed stroke is of the ischemic or hemorrhagic type.

13. The method according to claim 1, wherein the polypeptide has a molecular weight selected from the group consisting of about 4475, about 4634 and about 4797 Da, and a reduction, relative to a control, of a peak corresponding to the polypeptide is indicative of stroke.

14. The method according to claim 1, wherein the polypeptide has a molecular weight selected from the group consisting of about 6441 and about 6643 Da, and an increase, relative to a control, of a peak corresponding to the polypeptide is indicative of stroke.

15. The method according to claim 1, wherein the polypeptide has a molecular weight selected from the group consisting of about 11,530 and about 11,712 Da, and a reduction, relative to a control, of a peak corresponding to the polypeptide is indicative of stroke.

16. A kit for use in diagnosis of stroke, comprising a probe for receiving a sample of body fluid, and for placement in a mass spectrometer, thereby to determine a test amount of a polypeptide in the sample, wherein the polypeptide is differentially contained in the body fluid of stroke-affected subjects and non-stroke-affected subjects, and has a molecular weight selected from the group consisting of about 3900, about 3970, about 3990, about 6945, about 10,070, about 14,040, about 28,000, about 5920, about 6660, about 7770, about 4475, about 4634, about 4797, about 6441, about 6643, about 11,530 and about 11,712 Da.

17. The kit according to claim 16, in which the probe contains an adsorbent for adsorption of the polypeptide.

18. The kit according to claim 17, further comprising a washing solution for removal of unbound or weakly bound materials from the probe.
